(19) United States
(12) Patent Application Publication
(10) Pub. No.: US 2011/0091979 A1
(43) Pub. Date: Apr. 21, 2011

(54) Tracing Coalbed Natural Gas - Coproduced Water Using Stable Isotopes of Carbon

(75) Inventors: Shikha Sharma, Laramie, WY (US); Carol Frost, Laramie, WY (US)

(73) Assignee: UNIVERSITY OF WYOMING, Laramie, WY (US)

(21) Appl. No.: 12/921,661

(22) PCT Filed: Mar. 10, 2009
(86) PCT No.: PCT/US09/36695

§ 371 Date: Dec. 28, 2010

(30) Foreign Application Priority Data
Mar. 12, 2008 (US) 61/035831

(51) Int. Cl. G01N 33/18

(52) U.S. Cl. 436/56

(57) ABSTRACT

Water collected in the area of coal beds has strongly positive δ¹³C_{DIC} (12‰ to 22‰) that is readily distinguished from the negative δ¹³C of most surface and ground water (−8‰ to −11‰). Furthermore, the DIC concentrations in coproduced water samples are also high (more than 100 mg C/L) compared to the 20 to 50 mg C/L in ambient surface and ground water of the region. The distinctively high δ¹³C and DIC concentrations allow the identification of surface and ground water that have incorporated CBNG-coproduced water.

This application claims priority to U.S. Patent Application Ser. No. 61/035,831, filed Mar. 12, 2008, which is incorporated herein in its entirety by this reference.

This invention was made, at least in part, with the United States governmental support awarded by the U.S. Department of Energy Grant No. DE-FC26-06NT15568-Task 4. The United States Government has certain rights in this application.

### BACKGROUND OF THE INVENTION

The Powder River Basin in northeastern Wyoming is one of the most active areas of coalbed natural gas (CBNG) development in the western United States. This resource provides clean energy but raises environmental concerns. Primary among these is the disposal of water that is coproduced with the gas during depressurization of the coal seam. The Paleocene and Eocene coals of the Powder River Basin contain reserves estimated at more than 25 trillion cubic feet of methane. More than 22,000 CBNG wells have been drilled. Water production from individual wells varies, but on average more than 4600 gallons of water per well per day are produced (Wyoming Oil and Gas Commission Web site). The quality of the CBNG-coproduced water varies from high quality that meets state and federal drinking water standards to low quality due to high salinity and/or high sodicity. The higher quality water can be used to supplement area water supplies. However, if the water does not meet federal and state standards for beneficial use and the cost of treatment is uneconomical, the water can be disposed of by discharge into ponds and surface drainages where it will infiltrate into the shallow ground water or by reinjection into subsurface formations. In either case, we require a tool to identify and track the fate of the CBNG-produced water after its disposal. Standard geochemical characteristics of the CBNG-coproduced water are insufficient to distinguish CBNG-coproduced from subsurface or shallow ground water in the Powder River Basin, and therefore, Sr isotope ratios have been used to fingerprint the CBNG-coproduced water (Frost and Brinck 2005; Brinck and Frost 2007). However, significant Sr contribution from local lithologies to CBNG-coproduced water and high costs of Sr isotope analysis may limit the applicability of this technique.

### SUMMARY OF THE INVENTION

Recovery of hydrocarbons commonly is associated with coproduction of water. This water may be put to beneficial use or may be reinjected into subsurface aquifers. In either case, it would be helpful to establish a fingerprint for that coproduced water so that it may be tracked following discharge on the surface or reintroduction to geologic reservoirs. In this invention, δ¹³C of dissolved inorganic carbon (DIC) of coalbed natural gas (CBNG)-coproduced water is used as a fingerprint of its origin and to trace its fate once it is disposed on the surface. Water samples coproduced with CBNG from the Powder River Basin show that this water has strongly positive δ¹³C_{DIC} (12‰ to 22‰) that is readily distinguished from the negative δ¹³C of most surface and ground water (−8‰ to −11‰). Furthermore, the DIC concentrations in coproduced water samples are also high (more than 100 mg C/L) compared to the 20 to 50 mg C/L in ambient surface and ground water of the region. The distinctively high δ¹³C and DIC concentrations allow us to identify surface and ground water that have incorporated CBNG-coproduced water. Accordingly, the δ¹³C_{DIC} and DIC concentrations of water can be used for long-term monitoring of infiltration of CBNG-coproduced water into ground water and streams. Our results also show that the δ¹³C_{DIC} of CBNG-coproduced water from two different coal zones are distinct such that δ¹³C_{DIC} can be used to distinguish water produced from different coal zones.

### BRIEF DESCRIPTION OF THE FIGURES

FIG. 1 is the δ¹³C_{DIC} values of surface water samples collected from the Powder River and its tributaries. Note the trend of increasing δ¹³C_{DIC} values from sample PR7 and then a decrease from sample PR23 onward during both low-flow (2006) and high-flow (2007) conditions. The high values correspond to the region where CBNG production is concentrated. Inset on the upper left corner shows locations of surface water samples collected along the Powder River and its tributaries.

FIG. 2 is a graph showing correlation between DIC concentration values, δ¹³C_{DIC} values, and Ca concentrations in surface water samples collected from the Powder River and its tributaries during high-flow conditions of 2007.

FIG. 3 is the δ¹³C_{DIC} and DIC concentration and Ca concentration trend in water samples collected from the Beaver Creek site. BC-2 and BC-4 are ground water monitoring wells upstream of the CBNG discharge point UP-CBM. UPQ is the pond that holds the CBNG-coproduced water and BC-7 is a ground water monitoring well installed downstream of the pond. The location map of sampling sites is shown in the inset at the left upper corner.

### DESCRIPTION OF THE INVENTION

Measuring δ¹³C (which is the ¹³C/¹²C ratio expressed as per mil deviation from an international standard) of dissolved inorganic carbon (DIC) in ground water provides a low-cost diagnostic tool to trace water sources and to understand ground water interactions if there are large differences in δ¹³C values among different carbon reservoirs in a particular region. The δ¹³C of DIC is controlled by the isotopic composition of the carbon sources. The major sources of carbon contributing to DIC in natural ground water are CO₂ derived from root respiration or microbial decay of organic matter and the dissolution of carbonate minerals. CO₂ derived from root respiration or microbial decay of organic matter has δ¹³C centered around −25‰ in temperate climates where C3 plants dominate. After dissolution of this soil CO₂, the pH of infiltrating water decreases and is able to dissolve the soil carbonates with δ¹³C of approximately +1‰:

CO₂+H₂O+CaCO₃=>2HCO₃⁻+Ca²⁻

This process results in δ¹³C of the dissolved bicarbonate of about −12‰ (i.e., [−25+1]/2=−12) in temperate climates. This bicarbonate then undergoes isotope exchange with soil CO₂, and depending on the pH and concentration of the biogenic CO₂, the δ¹³C_{DIC} may acquire more negative values. For example, ground water in thickly vegetated drainage basins with soils of low carbonate contents can acquire δ¹³C_{DIC} values as negative as −26‰ (Mook and Tan 1999). Therefore, it seems logical to presume that subsurface water draining areas of moderate vegetation typically should have intermediate δ¹³C values of DIC that range from −12‰ to more negative values. The slightly higher observed values of −9‰±1‰ can be caused by the occurrence of rock weathering (carbonate δ¹³C=−2‰±2‰, and the highest δ¹³C_{DIC} values (+1±1 per thousand) in natural water are produced by isotopic equilibrium between the DIC fractions and the atmospheric CO₂ (8‰±1‰ in lakes or reservoirs where residence time of water is very long (Mook and Tan 1999). Higher or more positive δ¹³C_{DIC} (10‰±30‰ can only be recorded in organic-rich systems where bacteria preferentially removes ¹²C from the system during the process of microbial methanogenesis releasing isotopically light CH₄ (acetate fermentation ˜−40‰; CO₂ reduction ˜−70‰, leaving the remaining DIC in the formation water highly enriched in ¹³C (Simpkins and Parkin 1993, Botz et al. 1996; Taylor 1997; Whiticar 1999). Thus, in a closed system where either of these processes are taking place, the δ¹³C_{DIC} in CBNG reservoir will become increasingly isotopically enriched in ¹³C due to continued preferential removal of ¹²C from the carbon pool as methanogenesis progresses. Therefore, δ¹³C_{DIC} can prove to be a diagnostic tool for distinguishing water originating from coal aquifers in basins like the Powder River Basin where biogenic methanogenesis is the prime mechanism of methane generation (Gorody 1999; Rice 1993).

The concentration of DIC coupled with δ¹³C_{DIC} can be taken as an additional indicator of methanogenesis in subsurface water. As discussed earlier, two main processes contributing to the DIC in formation water are dissolution of carbonate rock and decay of organic matter. The increase in DIC concentration due to carbonate dissolution will be accompanied by increase in Ca²⁺ and slight increase in δ¹³C_{DIC} depending on the δ¹³C of the dissolving carbonate. In contrast, increase in DIC concentration due to organic matter degradation will be accompanied by either decreasing δ¹³C_{DIC} values in oxidizing environments or increasing δ¹³C_{DIC} values in reducing environments (Grossman et al. 1989; Ogrinc et al. 1997; Hellings et al. 2000). This is due to the fact that in oxidizing environments, the carbon in formation water is derived from respiration of organic matter, which has a lighter carbon isotope ratio compared to the original DIC resulting in decreasing δ¹³C_{DIC} values. However, in reducing environments, production of highly ¹³C-depleted methane (by acetate fermentation or CO₂ reduction) supplies ¹³C-enriched CO₂ to the system resulting in increasing δ¹³C values in formation water with increase in DIC concentration. Therefore, we hypothesize that in CBNG-coproduced water, the high DIC concentrations will be accompanied by higher δ¹³C_{DIC} values.

### Samples and Methods

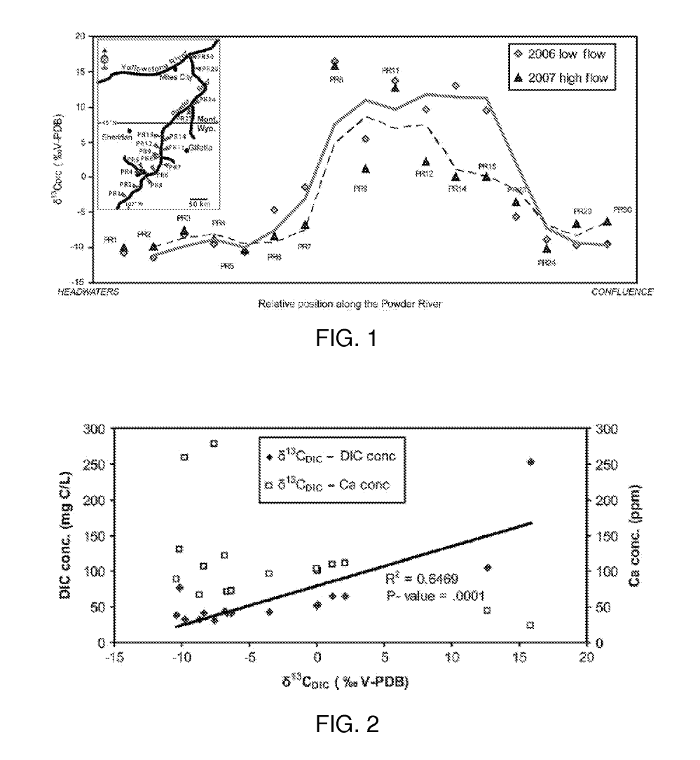

We analyzed three groups of water samples from the Powder River Basin of northeastern Wyoming and southeastern Montana as part of this study (see detailed location map, Table S1). First, we analyzed samples of coproduced water from CBNG wellheads in three different parts of the basin to observe if CBNG-coproduced water samples from different coal zones and different geographic locations have distinct δ¹³C_{DIC} signatures. These samples include water produced from five wells located southwest of Gillette completed in the Wyodak coal seam of the Upper Wyodak coal zone: two samples of water produced from the Wall coal of the Lower Wyodak coal zone in northeast Sheridan County west of the Powder River; and two samples from wells located northwest of Gillette, one completed in the Upper Wyodak and one in the Lower Wyodak coal zone. Second, we analyzed surface water samples from the Powder River and several tributaries to evaluate whether CBNG-coproduced water discharged to surface drainages can be traced isotopically into major river systems. Sampling along Powder River was done from its headwater west of Casper, Wyo., to its confluence with the Yellowstone River in Montana (inset, FIG. 1). The sampling took place from Sep. 21 to 24, 2006, a time when the river was near its lowest flow and between Jun. 30 to Jul. 4, 2007, when river was near high-flow conditions. The sample set includes 14 samples of the main stem of the Powder River and 3 samples from tributaries in Wyoming and Montana. The tributaries sampled are Beaver Creek (PR8), Flying E (PR11), and Little Powder River (PR24).

A third group of samples was collected from the headwater of Beaver Creek, a tributary of the Powder River. This includes samples from a standpipe that discharges coproduced water from a number of CBNG wells and from a retention pond into which this water is discharged, along with samples of the ambient shallow ground water from monitoring wells installed upgradient of this pond and a shallow monitoring well located within the ephemeral channel downgradient from the pond. These monitoring wells were installed by the Western Resources Project as part of a study of the effects of CBNG development on surface and shallow ground water systems in the Powder River Basin (Wheaton and Brown 2005; Payne and Saffer 2005; Frost and Brinck 2005).

Samples collected for DIC analyses were passed through a Cameo 0.45 μm nylon prefilter attached to 60 cc Luer-lock syringe. The water sample was then transferred in 30 mL Wheaton glass serum vials with Teflon® septa and sealed with A1 caps using a crimper. A few drops (two to three) of benzalkonium chloride were added to each vial before filling it with water to halt any metabolic activity. Samples were analyzed for δ¹³C_{DIC} on a GasBench-II device coupled to a Finnigan DELTA plus mass spectrometer in the central Stable Isotope Facility at the University of Wyoming. The reproducibility and accuracy were monitored by replicate analysis of samples and internal lab standards and was better than ±0.1‰. The δ¹³C_{DIC} values are reported in 3 per mil relative to V-PDB. The DIC concentrations in samples were also quantified from the mass spectrometry data. Three NaHCO₃ stock solutions of different DIC concentrations were prepared for this purpose. DIC concentrations were then quantified based on the peak areas of the mass 44-ion trace of these standards. Plotting peak area of CO₂ vs. concentration of DIC in these standards gives an excellent correlation (r²=0.995), indicating that DIC concentrations of the samples could be quantified using this method. The relative standard uncertainty of the DIC concentration measurement in this study was ±3%.

### Results and Discussion

The wellhead samples collected from different coal zones and different parts of the basin show positive δ¹³C_{DIC} values of +12‰ to +22‰ and high DIC concentrations of above 100 mg C/L (see Tables S1 and S2). The positive δ¹³C_{DIC} values reflect preferential removal of ¹²C from the carbon pool by the methanogens present in the formation water. The DIC concentrations are similar in the CBNG-coproduced water from both coal zones. However, the δ¹³C_{DIC} of the CBNG-coproduced water from the Upper Wyodak coal zone, which vary from +18.4‰ to +22.1‰, is 7‰ to 8‰ more enriched in δ¹³C_{DIC} than water being produced from the Lower Wyodak coal zone, which yielded δ¹³C_{DIC} of 12.2‰ to 14.3‰ (Tables S1 and S2). This difference in the δ¹³C_{DIC} values could reflect differing conditions under which methanogenesis is taking place and/or the reaction progress/degree of methanogenesis in these coal zones or the Lower Wyodak water might be affected by leakage of ground water from other aquifers with lower δ¹³C_{DIC} values.

The samples collected along the length of Powder River also show a range of δ¹³C_{DIC} values (FIG. 1). During the 2006 collection period, the samples from 4 South, Middle, and North Forks of the Powder River (PR1 to 5) upstream of CBNG development have δ¹³C_{DIC} values between −8.3‰ and −11.4‰, Samples collected near Sussex and Fort Reno, Wyo. (PR6 and 7), have δ¹³C_{DIC} that are less negative (−4.7‰ and −1.4‰). These values may reflect incorporation of CBNG water discharged from production in this area. Downstream of these samples is an area of more intense CBNG development, including the Beaver Creek drainage, which receives significant coproduced water discharge. The δ¹³C_{DIC} of water from Beaver Creek (PR8) is +16.4‰, which is within the range of δ¹³C_{DIC} that we analyzed of CBNG-coproduced water directly from wellheads. It appears that in the fall, the water in the Beaver Creek tributary is dominated by CBNG discharge. The highly positive δ¹³C_{DIC} of Powder River samples in Wyoming downstream from Beaver Creek (PR9 to 15) suggests the presence of CBNG-produced water in the river related to local CBNG production. The Powder River samples collected in Montana all have negative d13CDIC. Only sample PR23, from the Powder River at Broadus, Mont., has δ¹³C_{DIC} (−5.58‰) above the ambient value of approximately −10‰. This suggests that surface water in Montana is little to unaffected by CBNG production during the low-flow conditions. A second set of samples were collected in June 2007 during high-flow conditions. The 2007 samples also show broadly the same trend; that is, samples from the stretch of Powder River passing through the area of CBNG development (samples PR8 to 15) have higher δ¹³C_{DIC} values than does river water upstream and downstream (FIG. 1). However, the δ¹³C_{DIC} of the Powder River samples at high flow are not as strongly positive as during low flow, reflecting the greater proportion of water from snowmelt during the spring runoff. It is noteworthy that the δ¹³C_{DIC} of Beaver Creek (PR8) and Flying E (PR11) tributaries does not appear to vary seasonally. These tributaries drain small catchments within the basin that do not accumulate significant snowpack; hence, their discharge does not show the same variation from spring to fall as characterizes the main stem of the Powder River.

The δ¹³C_{DIC} of Powder River samples shows a significant correlation (R²=0.65 and p=0.0001) with DIC concentration and the samples with high δ¹³C_{DIC} values have higher DIC concentrations (FIG. 2). However, the δ¹³C_{DIC} values do not show a significant correlation with Ca concentrations (R²=0.22 and p=0.06) as depicted in FIG. 2. This indicates that higher DIC concentrations are due to considerable contribution of methanogenic water (with higher δ¹³C_{DIC} values) to the flow in areas affected by CBNG development. We plan to continue our monitoring and to increase our sample density in the coming years to verify these preliminary results and document any future changes that may occur. In any case, the results of this preliminary investigation demonstrate the value of using δ¹³C_{DIC} as a tracer for CBNG-coproduced water in the surface water and should be an effective tool for monitoring and guiding water quality regulatory issues in the region.

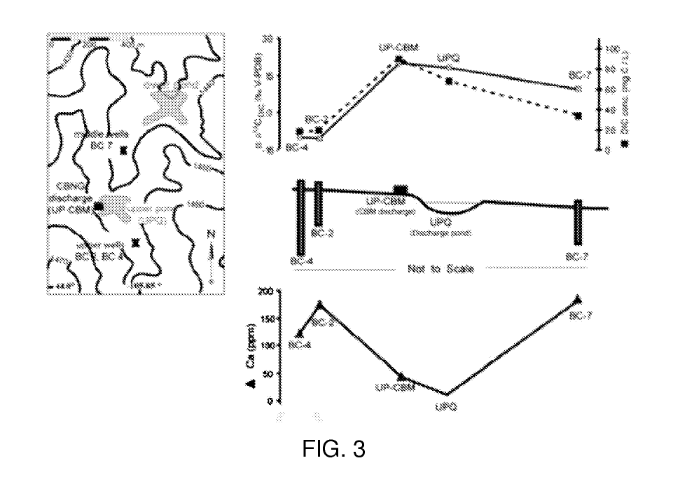

The ambient shallow ground water samples collected from the two upgradient monitoring wells at Beaver Creek, BC-2 and BC-4, show low δ¹³C_{DIC} values of −10.3‰ and −10.0‰, respectively (FIG. 3). These are within the range of expected values for subsurface water in most natural systems. In contrast, water samples collected from the CBNG discharge point (UP-CBM) and the corresponding CBNG-produced water retention pond (UPQ) yielded values of +19.8‰ and +17.8‰, respectively, within the range of δ¹³C_{DIC} for the coproduced water samples discussed previously. The water from the shallow ground water monitoring well below the retention pond at Beaver Creek (BC-7) shows a δ¹³C_{DIC} value of +9.3‰, intermediate between the values of ambient ground water and CBNG-coproduced water (FIG. 3). Brinck and Frost (2007) used ⁸⁷Sr/⁸⁶Sr ratios and Sr concentrations of these same samples to calculate that a minimum of 70% of the water in monitoring well BC-7 originated from the CBNG discharge. The intermediate δ¹³C_{DIC} value of this water also suggests a mixed system containing both CBNG water and ambient water. Although complicated by processes of carbonate dissolution and precipitation, the proportions of each endmember suggested by the δ¹³C_{DIC} values (approximately two-thirds CBNG, one-third ambient ground water) is similar to the proportions calculated from Sr isotopic data. The DIC concentrations are also high in the UP-CBM (CBNG discharge point) and UPQ (retention pond) samples (FIG. 3) compared to other samples. The high DIC concentrations do not appear to be related to higher CaCO₃ dissolution from source rocks because the two samples showing the highest DIC concentration (UP-CBM and UPQ) have the lowest Ca concentrations (Brinck and Frost 2007). Therefore, the high DIC concentration in these samples is also indicative of contribution of methanogenic processes to the DIC.

### Conclusions

Our initial results demonstrate that δ¹³C of DIC and DIC concentration in coproduced CBNG water is distinct from shallow ground water and surface water in Powder River Basin. Moreover, the δ¹³C_{DIC} of two different coal zones are distinct, leading to the possibility of using δ¹³C_{DIC} to fingerprint water produced from different coal seams. A monitoring well containing a mixture of ambient shallow ground water and infiltrating CBNG-coproduced water yielded an intermediate δ¹³C_{DIC} that suggested proportions of each endmember consistent with the fractions calculated from Sr isotopic mass balance. Our study establishes δ¹³C_{DIC} and DIC concentration as a powerful fingerprint for tracing CBNG on the surface and subsurface and makes it possible to monitor the fate of CBNG-coproduced water into ground water and streams of the region.

### Supplementary Material

The following supplementary materials are available for this article: Table S1. δ¹³C_{DIC}, DIC concentration, Ca concentration and location details of samples collected from different parts of Powder River basin; Table S2. δ¹³C_{DIC} and DIC concentration in water samples collected from well heads producing water from two different coal zones of Powder River Basin. Filled symbols=Upper Wyodak coal zone; Open symbols=Lower Wyodak coal zone.

This material is available as part of the online article from: http://www.blackwell-synergy.com/doi/abs/10.1111/j.1745-6584.2007.00417.x, which is incorporated herein in its entirety by this reference.

### REFERENCES

Botz, R., H. D. Polojski, M. Schmitt, and M. Thomm. 1996. Carbon isotope fractionation during bacterial methanogenesis by CO2 reduction. Organic Geochemistry 25, no. 3: 255-262.
Brinck, E. L., and C. D. Frost. 2007. Detecting infiltration and impacts of introduced water using strontium isotopes. Ground Water 45, no. 5: 554-568.
Frost, C. D., and E. Brinck. 2005. Strontium isotope tracing of the effects of coal bed natural gas (CBNG) development on shallow and deep groundwater systems in the Powder River Basin, Wyo. In Western Resources Project Final Report—Produced Groundwater Associated with Coalbed Natural Gas Production in the Powder River Basin. Wyoming State Geological Survey Report of Investigations No. 55, ed. M. D.
Zoback, 93-107. Laramie, Wyo.: WyGS.
Gorody, A. W. 1999. The origin of natural gas in the Tertiary coal seams on the eastern margin of the Powder River Basin. In Coalbed methane and Tertiary geology of the Powder River Basin: Wyoming Geological Association Guidebook, 50th Annual Field Conference, ed. W. R. Miller, 7 89-101.
Grossman, E., B. K. Coffman, S. J. Fritz, and H. Wada. 1989. Bacterial production of methane and its influence on ground-water chemistry in east-central Texas aquifers. Geology 17, no. 6: 495-499.
Hellings, L., V. D. K. Driessche, W. Baeyens, E. Keppens, and F. Dehairs. 2000. Origin and fate of dissolved inorganic carbon in interstitial waters of two freshwater intertidal areas: A case study of the Scheldt Estuary, Belgium. Biogeochemistry 51, no. 2: 141-160.
Mook, W. G., and F. C. Tan. 1991. Stable carbon isotopes in rivers and estuaries. In Biogeochemistry of Major World Rivers, ed. E. T. Degens, S. Kempe, and J. E. Richey, 245-264. Chichester, UK: John Wiley and Sons.
Ogrinc, N., S. Lojen, and J. Faganeli. 1997. The sources of dissolved inorganic carbon in pore waters of lacustrine sediment. Water, Air and Soil Pollution 99, no. 1-4: 333-341.
Payne, A. A., and D. M. Saffer. 2005. Surface water hydrology and shallow groundwater effects of coalbed natural gas development, upper Beaver Creek drainage, Powder River Basin, Wyo. In Western Resources Project Final Report—Produced Groundwater Associated with Coalbed Natural Gas Production in the Powder River Basin. Wyoming State Geological Survey Report of Investigations No. 55, ed. M. D.
Zoback, 5-43. Laramie, Wyo.: WyGS.
Rice, D. D. 1993. Composition and origins of coalbed gas. In Hydrocarbons from Coal: American Association of Petroleum Geologists Studies in Geology 38, ed. B. E. Law and D. D. Rice, 159-184.8
Simpkins, W. W., and T. B. Parkin. 1993. Hydrogeology and redox geochemistry of CH4 in a Late Wisconsin till and loess sequence in central Iowa. Water Resources Research 29, no. 11: 3643-3657.
Taylor, C. B. 1997. On the isotopic composition of dissolved inorganic carbon in rivers and shallow groundwater: A diagrammatic approach to process identification and a more realistic model of the open system. Radiocarbon 39, no. 3: 251-269.
Wheaton, J., and T. H. Brown. 2005. Predicting changes in groundwater quality associated with coalbed natural gas infiltration ponds. In Western Resources Project Final Report—Produced Groundwater Associated with Coalbed Natural Gas Production in the Powder River Basin. Wyoming State Geological Survey Report of Investigations No. 55, ed. M. D. Zoback, 45-69. Laramie, Wyo.: WyGS. 9
Whiticar, M. J. 1999. Carbon and hydrogen isotope systematics of bacterial formation and oxidation of methane. Chemical Geology 161, no. 1: 291-314.
Wyoming Oil and Gas Commission Web site. http://wogcc.state.wy.us

The foregoing description and drawings comprise illustrative embodiments of the present invention. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A method for identifying coal bed natural gas co-produced water, comprising measuring in a water sample a parameter selected from the group consisting of δ¹³C_{DIC} and dissolved inorganic carbon.

2. The method of claim 1, wherein the parameter is δ¹³C_{DIC} and it is greater than about +10%.

3. The method of claim 1, wherein the parameter is dissolved inorganic carbon and it is greater than about 100 mg C/L.

4. A method for tracing the flow of, comprising measuring in a water sample taken in a first location a parameter selected from the group consisting of δ¹³C_{DIC} and dissolved inorganic carbon and correlating it to a known sample of coal bed natural gas co-produced water from a second, source location.

5. The method of claim 4, wherein the parameter is δ¹³C_{DIC} and it is greater than about +10%.

6. The method of claim 4, wherein the parameter is dissolved inorganic carbon and it is greater than about 100 mg C/L.
